(19) United States
(12) Patent Application Publication
(10) Pub. No.: US 2025/0388960 A1
(43) Pub. Date: Dec. 25, 2025

(54) COMPOSITION FOR DETECTING CDNA SYNTHESIS-BASED TARGET GENE USING LIGATION METHOD THAT DOES NOT USE REVERSE TRANSCRIPTION, AND METHOD FOR AMPLIFYING MULTIPLE LIGATION-ASSISTED RECOMBINASE POLYMERASE

(71) Applicant: INDUSTRIAL COOPERATION FOUNDATION JEONBUK NATIONAL UNIVERSITY, Jeonju-si, Jeollabuk-do (KR)

(72) Inventors: Young Jun SEO, Jeonju-si, Jeollabuk-do (KR); Moon Hyeok CHOI, Jeonju-si, Jeollabuk-do (KR)

(21) Appl. No.: 18/842,743

(22) PCT Filed: Mar. 20, 2023
(86) PCT No.: PCT/KR2023/003648

§ 371 Date: Aug. 29, 2024

(30) Foreign Application Priority Data
Mar. 24, 2022 (KR) 10-2022-0036576

(51) Int. Cl. C12Q 1/6862; C12Q 1/48; C12Q 1/6809; C12Q 1/6876; C12Q 1/70

(52) U.S. Cl. CPC C12Q 1/6862; C12Q 1/48; C12Q 1/6809; C12Q 1/6876; C12Q 1/701; C12Y 207/07; C12Y 600/00

(57) ABSTRACT

The present invention relates to a composition for detecting a target gene based on cDNA synthesis using a ligation method that does not use reverse transcription and a method for multiple ligation-assisted recombinase polymerase amplification, and since a target gene may be detected through a visual change with only a short reaction time of about 30 minutes at room temperature without the synthesis of cDNA using reverse transcriptase, the present invention may be effectively used for point-of-care genetic molecular diagnosis of RNA viruses and the like.

### TECHNICAL FIELD

The present invention relates to a composition for detecting target gene based on cDNA synthesis using a ligation method that does not use reverse transcription and a method for multiple ligation-assisted recombinase polymerase amplification (mLig-RPA).

### BACKGROUND ART

Viruses are fatal because they can spread and infect their targets rapidly. Many dangerous viruses have their own RNA genomes that easily cause mutations. Novel severe acute respiratory syndrome coronavirus 2 (SARS-CoV-2) is a new type of coronavirus that was first detected at the end of 2019 and remains spreading to date. Such viruses that are highly infectious can result in infectious diseases. Therefore, it is important to rapidly diagnose viruses.

The reverse transcription-based polymerase chain reaction (RT-PCR) is commonly used for the detection of viral RNA because it has proven its ability to provide sensitive and selective detection of viral genomes. Nevertheless, it has several drawbacks: it is time-consuming and demands expensive instrumentation, and thus it is difficult to be used for point-of-care. Hence, many alternative methods have been developed for simple and rapid point-of-care detection. Viral RNA detection has been implemented by recombinase polymerase amplification (RPA), rolling circle amplification (RCA), and loop-mediated isothermal amplification (LAMP). Nevertheless, some of isothermal amplification-based point-of-care detection systems have a limitation because they operate with low selectivity compared to the RT-PCR method for the diagnosis of viral RNA.

Meanwhile, the LAMP system generally requires reverse transcriptase (RT) to produce target cDNA using a primer sequence that binds to viral RNA. However, since many erroneous forms of cDNA are produced during the process of reverse transcription using the primer, problems described below may occur. The first problem is the possibility of false-positive diagnosis. When extracting the actual target viral RNA, there are the possibility of the existence of other similar viral RNAs, and the possibility that some sequences in the human genome are similar, and in this case, it may be difficult for the primer to distinguish among them. In addition, there is the possibility of false-positive detection even when there is no actual target RNA. Therefore, it is important to design a primer with a target that does not overlap with areas any other. Thus, most primers have targeted a conservative site, such as the nucleoprotein, during RT-LAMP. However, when the primer sequence is excessively specific, it tends to become difficult to bind to the target RNA, and thus there is the possibility of false-negative detection. In other words, even when the actual target RNA is present, there may be cases where the primer may not bind thereto and, thus, the target cDNA is not be formed.

Therefore, in order to overcome the selectivity problem of LAMP, the present inventors conducted research to develop a method for amplifying viral RNA without using reverse transcriptase for application to the LAMP process and completed the present invention.
(Patent Document 1) Korean Patent Publication No. 10-2294552

### DETAILED DESCRIPTION OF THE INVENTION

### Technical Problem

An object of the present invention is to provide a composition for detecting a target gene, including: a plurality of template sequences complementary to a part of a base sequence of a target gene; a ligase; a loop-mediated isothermal amplification reagent; and a primer set for loop-mediated isothermal amplification, wherein the plurality of template sequences are complementary to the entire base sequence of the target gene, when all of the plurality of template sequences are ligated.

Another object of the present invention is to provide an information providing method for determining the presence of a target gene from a subject, including: obtaining a biological sample from a subject; performing multiple ligation of template sequences by adding a plurality of template sequences complementary to a part of a base sequence of a target gene and a ligase to the sample; performing an amplification reaction of the multiple ligated template sequences; and determining that the target gene is present in the subject when the amplification reaction of the template sequences is confirmed.

### Technical Solution

An aspect of the present invention is to provide a composition for detecting a target gene, including: a plurality of template sequences complementary to a part of a base sequence of a target gene; a ligase; a loop-mediated isothermal amplification reagent; and a primer set for loop-mediated isothermal amplification, wherein the plurality of template sequences are complementary to the entire base sequence of the target gene, when all of the plurality of template sequences are ligated.

In order to overcome the selectivity problem of loop-mediated isothermal amplification (LAMP), the present invention provides a dual-site ligation-assisted cDNA synthesis method that does not use reverse transcriptase for application to the LAMP process. In other words, by using a ligase together with a plurality of DNA oligonucleotide template sequences complementary to a specific region of a target RNA sequence to link the template sequences, cDNA may be produced within a few minutes, and the production of the cDNA occurs only in the presence of the target RNA. The produced cDNA is then amplified by a LAMP reaction system. Therefore, according to the present invention, when there is a sequence that does not match a target gene or when one of the ligation templates does not bind, cDNA synthesis and amplification reactions do not occur, and so the presence of a target gene may be rapidly detected using the composition for detecting a target gene of the present invention. Therefore, the composition for detecting a target gene of the present invention can be effectively used to solve the selectivity problem that occurs when an isothermal amplification method is used.

According to one embodiment of the present invention, the number of the plurality of template sequences may be 2 to 10.

The number of the plurality of template sequences may be 2 to 10, more preferably 2 to 7, and most preferably 3.

According to one embodiment of the present invention, the target gene may consist of 20 to 200 base sequences.

The target gene may consist of 20 to 200 base sequences, more preferably 40 to 160 base sequences, further preferably 60 to 100 base sequences, and most preferably 76 to 77.

According to one embodiment of the present invention, the ligase may be Splint R ligase.

Splint R ligase is an RNA-templated DNA ligase that has a much faster reaction rate than T4 DNA ligase, and thus may effectively perform ligation of a plurality of template sequences complementary to a part of a base sequence of a gene.

According to one embodiment of the present invention, the primer set for LAMP may consist of SEQ ID NOs: 1 to 6.

According to one embodiment of the present invention, the plurality of template sequences complementary to a part of a base sequence of a target gene may consist of SEQ ID NOs: 7 to 9.

According to one embodiment of the present invention, the target gene may be a SARS-CoV-2 virus-derived base sequence.

When a primer set for LAMP consisting of SEQ ID NOs: 1 to 6 and a template sequence consisting of SEQ ID NOs: 7 to 9 are used, a SARS-CoV-2 virus-derived RNA consisting of SEQ ID NO: 10 may be detected with high sensitivity and selectivity, and thus they may be effectively used for rapid point-of-care of a SARS-CoV-2 virus.

According to one embodiment of the present invention, the SARS-CoV-2 virus-derived base sequence may be SEQ ID NO: 10.

According to one embodiment of the present invention, the composition may further include a compound represented by Chemical Formula 1 below:

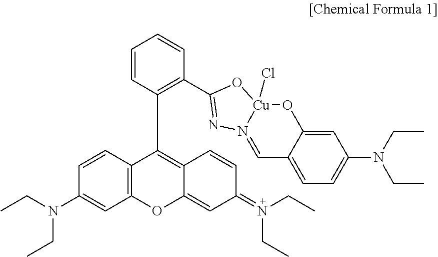

The conventional method for diagnosing genes uses multiple oligonucleotides that have undergone a complex synthetic process and a method of amplifying signals with fluorescence that is tagged to a gene. However, since these methods consume a lot of time and costs and require a fluorescence analyzer, they were unsuitable for rapid point-of care.

The compound represented by Chemical Formula 1 of the present invention not only selectively has a high affinity to pyrophosphate, which is inevitably released in various nucleic acid amplification reactions, but also allows for visual detection of changes, and thus may be utilized as a colorimetric detector for analyzing in real time whether a nucleic acid amplification reaction has occurred in combination with a nucleic acid amplification method.

The composition of the present invention may further include a reagent necessary for LAMP. For example, the reagent may include a buffer, a DNA polymerase, a DNA polymerase cofactor, a uracil-DNA glycosylase (UDG), and/or deoxyribonucleotide triphosphates (dNTPs) but are not limited thereto, and may be easily selected by one of ordinary skill in the art.

Another aspect of the present invention provides an information providing method for determining the presence of a target gene from a subject, including: obtaining a biological sample from a subject; performing multiple ligation of template sequences by adding a plurality of template sequences complementary to a part of a base sequence of a target gene and a ligase to the sample; performing an amplification reaction of the multiple ligated template sequences; and determining that the target gene is present in the subject when the amplification reaction of the template sequences is confirmed.

To avoid excessive complexity due to unnecessary repetition in the present specification, description of common items is omitted.

According to the information providing method of the present invention, the reverse transcription process used for genetic diagnosis of RNA viruses is eliminated, and instead, cDNA synthesis and isothermal gene amplification are performed using ligation of a plurality of template sequences. When a base sequence of a target gene differs by 1-mer or more, some or all of the plurality of template sequences are not ligated, so that cDNA is not formed. Therefore, rapid point-of care molecular diagnosis (self-diagnosis) of RNA viruses with high sensitivity and selectivity is possible by the amplification reaction of the cDNA.

According to one embodiment of the present invention, the number of the plurality of template sequences may be 2 to 10.

According to one embodiment of the present invention, the target gene may consist of 20 to 200 base sequences.

According to one embodiment of the present invention, the multiple ligation may be performed by Splint R ligase.

According to one embodiment of the present invention, the amplification reaction may be LAMP.

According to one embodiment of the present invention, the plurality of template sequences may consist of SEQ ID NOs: 7 to 9.

According to one embodiment of the present invention, the target gene may be a SARS-CoV-2 virus-derived base sequence.

According to one embodiment of the present invention, the SARS-CoV-2 virus-derived base sequence is SEQ ID NO: 10.

According to one embodiment of the present invention, confirmation of the amplification reaction may be performed by confirming change in the color of an amplification product before or after the amplification reaction by adding a compound represented by Chemical Formula 1 below:

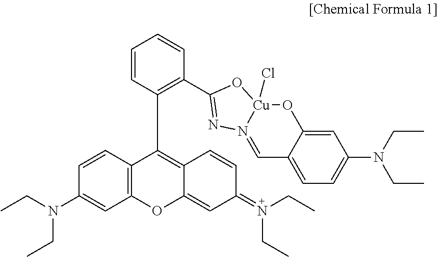

A biological sample, for which it is unknown whether a target gene is included therein, is amplified together with a compound represented by Chemical Formula 1 and a reagent necessary for amplification of a target nucleic acid. When the sample includes the target gene, an excess of pyrophosphate is produced during the nucleic acid amplification process, and when the target gene is not included, nucleic acid amplification does not occur, so there is no significant change in the pyrophosphate content in the sample.

Since the compound represented by the chemical formula 1 of the present invention exhibits selective affinity to pyrophosphate, when a target gene is present in a biological sample, the pyrophosphate produced during or after a nucleic acid amplification reaction reacts with the compound represented by the chemical formula 1. Meanwhile, when the compound represented by the chemical formula 1 reacts with pyrophosphate, Cu²⁺ ions are replaced, and the absorbance decreases due to the closure of a rhodamine ring, so that the color of the sample, which was pink before the reaction, becomes colorless after the reaction. Therefore, when a target gene is present in a biological sample, the color of the sample changes to colorless during or after a nucleic acid amplification reaction of the sample, so that it can be easily determine whether the target gene is present in the biological sample by visually recognizing the color change.

### Advantageous Effects

As described above, according to the composition for detecting a target gene based on cDNA synthesis using a ligation method that does not use reverse transcription and the information providing method for determining the presence of a target gene, a target gene may be detected through a visual change with only a short reaction time of about 30 minutes at room temperature without the synthesis of cDNA using reverse transcriptase, and therefore, the present invention may be effectively used for point-of-care genetic molecular diagnosis of RNA viruses and the like.

### DESCRIPTION OF THE DRAWINGS

FIG. 1 shows a conceptual diagram of dLig-LAMP and RT-LAMP assays. The diagram shows mechanism of PP probe sensing through the recognition of pyrophosphate and LAMP primer binding sites.

FIG. 2 shows a photograph and a graph of primer- and template-free negative control experiments using dLig-LAMP: (a) results of 20% PAGE; (b) colorimetric detection assay of samples 1 to 8 using a PP probe; and (c) absorbance spectra of samples 1 to 8 when using PP probe.

FIG. 3 shows a graph illustrating the sensitivity of the dLig-LAMP system: (a) absorption spectra of the dLig-LAMP reaction mixtures; and (b) linear relationship between the absorbance at 555 nm and the logarithm of the concentration of the target RNA.

FIG. 4 shows a graph illustrating the selectivity of the dLig-LAMP: (a) PAGE results of the RT-LAMP and the dLig-LAMP; (b) colorimetric detection using dLig-LAMP and the PP probe; and (c) absorbance of the PP probe in dLig-LAMP reaction.

FIG. 5 shows a photograph and a graph illustrating the results of the multiple ligation-assisted LAMP reaction: (a) colorimetric detection through the recognition of pyrophosphate in samples 1 and 2 using PP probe; and (b) absorbance spectra of samples 1 and 2 in the presence of a target negative control.

FIG. 6 shows (a) sensitivity of dLig-LAMP assays performed using full-genome SARS-CoV-2 at concentrations ranging from 8 to 1000 copies/reaction (copies/rxn) and (b) selectivity of the dLig-LAMP assay toward full-genome SARS-CoV-2, relative to various bacterial genomes, in terms of absorbance at 555 nm.

FIG. 7 shows (a) to (c) fluorescence spectra showing the sensitivity of dLig-LAMP assays performed using full-genome SARS-CoV-2 at concentrations ranging from 8 to 1000 copies/reaction (copies/rxn) and (b) linear relationship in a three-repeat study.

FIG. 8 shows a photograph and a graph illustrating the results of clinical validation of the dLig-LAMP/PP Probe system: (a) results of colorimetric detection assays, and (b) a graph illustrating the absorbance at 555 nm.

### BEST MODE

Hereinafter, the present invention will be described in more detail through one or more examples. However, these examples are intended to exemplify the present invention and the scope of the present invention is not limited to these examples.

### 1. Experimental Method

### 1-1 General Information

1-2 All DNA oligonucleotides and the dNTP mixture (dATP, dTTP, dCTP, dGTP) were purchased from Bioneer Corporation and Cosmo Genetech Co. Ltd. (South Korea). Target RNA and mismatched target RNA were synthesized using in vitro transcription. Splint R Ligase, WarmStart RTx Reverse Transcriptase, and Bst 2.0 WarmStart DNA polymerase were obtained from New England Biolabs (USA). PP (pyrophosphate-sensing) probe was prepared according to a previously reported procedure (Analytica Chimica Acta, 1176, 338765), and its spectra were in accordance with those described. UV-Vis absorption spectra were recorded using a Shimadzu (Japan) UV-1650PC spectrophotometer. Fluorescence was recorded using the PF-6500 spectrofluorometer (JASCO, Japan). All optical measurements were performed at room temperature, using a quartz cuvette (path length: 1 cm).

All gel electrophoresis was performed in 20% polyacrylamide gel (PAGE). 40% acrylamide/Bis solution 29:1 (purchased from Bio-Rad, USA; 15 mL), 10×tris-borate-ethylene-diamine-tetraacetic acid (TBE) buffer (3 mL), and 20% ammonium persulfate solution (dissolved in H₂O; 300 mL) were mixed in one tube, and water was added to a total volume of 30 mL. Tetramethylethylenediamine (TEMED) was added to make 20% polyacrylamide gel. The gel was loaded in an electrophoresis instrument (CBS Scientific, CA, USA) and treated at 180 V for 14 h. The gel was stained in an ethidium bromide (EtBr) solution for 10 minutes, and the stained gel was washed with water for 30 minutes. The gel photographs and colorimetric detection images were captured with a mobile device under a transilluminator.

### 1-2. DLig-LAMP Reaction

The dLig-LAMP reaction was performed with a total solution volume of 20 μL. A LAMP primer mixture was prepared including 16 μM of FIP/BIP primers, 2 μM of F3/B3 primers, and 4 μM of LF/LB primers. A cDNA template mixture including 10 nM of the LT-1/LT-2/LT-3 templates was prepared. For one dLig-LAMP reaction, the LAMP primer mixture (2 μL), the cDNA template mixture (2 μL), 10×isothermal amplification buffer [200 mM of Tris-HCl, 100 mM of (NH₄)₂SO₄, 500 mM of KCl, 20 mM of MgSO₄; pH 8.8 at 25° C.; 2 μL], 10×Splint R ligase buffer (500 mM of Tris-HCl, 100 mM of MgCl₂, and 10 mM of ATP; pH 7.5 at 25° C.; 2 μL), and a dNTP mixture (2 mM of dATP, dCTP, dGTP, and dTTP; 5 μL) were added into a 1.5-mL tube, and a target (5 μL) was added. Finally, the enzymes, which were Splint R ligase (25 U/μL; 1 μL) and Bst 2.0 WarmStart DNA polymerase (8 U/μL; 1 μL), were added into the reaction. The resulting mixture was cultured for 15 minutes at 37° C. and then cultured for 45 minutes at 65° C.

1-3. Primer and Template Negative Controls of dLig-LAMP

The dLig-LAMP reactions for the negative controls were performed using the standard procedure, except for one of the primer or templates. All reactions were monitored using PAGE. The colorimetric detection buffer (30% of 10 mM HEPES buffer and 70% acetonitrile; 180 μL) was added into the dLig-LAMP mixture to prepare a total volume of 200 μL for the colorimetric detection assay. A PP probe (25 mM, 1 μL) was added into a reaction tube, which was then shaken for one minute, and for detailed analysis, absorbance of the reactions performed in the presence of the PP probe was measured.

### 1-4. Sensitivity and Selectivity Measurements

The dLig-LAMP reactions were performed using a standard procedure. For sensitivity measurements, solutions of a target RNA were prepared with concentrations ranging from 1 aM to 1 nM. Reactions were performed in triplicate to determine the reproducibility, and the sensitivity was measured in terms of absorbance. For selectivity measurements, three different targets (matched target, one-base-mismatched target, and two-base-mismatched target) were used. The PAGE results were compared with those obtained using the RT-LAMP assay. One RT-LAMP reaction included a LAMP primer mixture (2 μL), a 10×isothermal amplification buffer [200 mM of Tris-HCl, 100 mM of (NH₄)₂SO₄, 500 mM of KCl, 20 mM of MgSO₄; pH 8.8 at 25° C.; 2 μL], a dNTP mixture (2 mM of dATP, dCTP, dGTP, and dTTP; 5 μL), water (4 μL), a target (5 μL), WarmStart RTx Reverse Transcriptase (15 U/μL; 1 μL), and Bst 2.0 WarmStart DNA polymerase (8 U/μL; 1 μL). The RT-LAMP reaction mixture was incubated at 65° C. for one hour. Each reaction was performed in triplicate to determine the reproducibility of the dLig-LAMP assays, and the selectivity was measured in terms of absorbance.

### 1-5. Multiple Ligation-Assisted LAMP Reaction

Short-ligation template mixtures were prepared to include 10 nM of LTs-1, LTs-2, LTs-3, LTs-4, LTs-5, LTs-6, LTs-7, LTs-8, LTs-9, LTs-10, and LTs-11. For comparison, one template-mismatched short-ligation template mixture including 10 nM of LTs-1, LTs-2, LTs-3, LTs-4, LTs-5, LTs-6 mismatch, LTs-7, LTs-8, LTs-9, LTs-10, and LTs-11 was prepared. For one multiple ligation-assisted LAMP reaction, a LAMP primer mixture (2 μL) and the short-ligation template mixture or one template-mismatched short-ligation template mixture (2 μL) were added. All the other protocols were carried out in the same manner as those for the dLig-LAMP process. The target RNA concentration was 1 nM. All reactions were confirmed using PAGE. A colorimetric detection buffer (30% of 10 mM HEPES buffer and 70% acetonitrile; 180 μL) was added into the dLig-LAMP reaction mixture to provide a total volume of 200 μL for the colorimetric detection assay. The PP probe (25 mM, 1 μL) was added into the reaction tube, which was then shaken for one minute. For detailed analysis, absorbance of the reactions performed in the presence of PP probe was measured.

### 1-6. Full-Genome SARS-CoV-2 Sensitivity and Bacteria Genome Selectivity Study

An AccuPlex™ SARS-CoV-2 Reference Material Kit (Seracare, Milford, MA, USA), which was assigned as 5000 copies/mL, was used for spiked samples. SARS-CoV-2 RNA was extracted using eMAG (BioMerieux, MarcylEtoile, France), according to the extraction protocol provided by the manufacturer, with an input volume of 200 μL and an elution volume of 50 μL. The copy concentration of the extracted RNA was approximately 20 copies/μL. To increase the concentration, the SARS-CoV-2 RNA was lyophilized to provide a concentration of 200 copies/μL. For the sensitivity study, the RNA sample was diluted in water to provide a concentration ranging from 1.6 to 200 copies/μL. The absorbance of all sample was measured using the dLig-LAMP system and the PP probe. A linear plot was calculated to obtain the limit of detection (LOD). For the selectivity study, samples of nine types of bacterial genomes, which are normal flora in the upper respiratory tract, were prepared. All bacterial DNA samples were extracted by a boiling method using a DNA extraction buffer (Seegene Inc., Seoul, South Korea). The extracted bacterial genomes were tested using the dLig-LAMP assay and the PP probe and compared with the SARS-CoV-2 genome. For detailed analysis, absorbance and fluorescence of the reactions performed in the presence of the PP probe were measured.

### 1-7. Clinical Sample Preparation and Validation

This study was approved by the Jeonbuk National University Hospital Institutional Review Board (CUIH 2021-11-005). Real-time reverse-transcription PCR (rRT-PCR) with a total of 40 residual samples for SARS-CoV-2 and five other virus samples (Influenza A Virus, Influenza B Virus, Respiratory Syncytial Virus A, Respiratory Syncytial Virus B, and Human Rhinovirus) was enrolled in this test: 20 positive samples, 20 negative samples, and 5 other virus samples. The samples were obtained from a nasopharyngeal swab collected in an eNAT tube (Copan Italy, Brescia, Italy) and stored at −20° C. after clinical tests. The nucleic acid was extracted with Magna Pure 24 (Roche Diagnostics, Basel, Swiss) or eMAG (BioMérieux, Marcy-l'Étoile, France), using the manufacturer's protocol. The rRT-PCR tests were performed using Allplex SARS-CoV-2 Assay (Seegene Inc., Seoul, South Korea). The cycle threshold (Ct) values of the positive samples ranged from 22.33 to 36.15 in the N gene. For clinical validation, 40 reactions for SARS-CoV-2 tests and 6 reactions for selectivity tests were performed using the dLig-LAMP reaction with the PP probe. For detailed analysis, absorbance of the reactions performed in the presence of the PP probe was measured.

Among them, the LT2 and LT3 templates presented a monophosphate unit at the 5′-end for the ligation event. Templates containing a phosphate modification at the 5′-end also allow the ligation by Splint R ligase to occur in the presence of a perfectly matched target RNA.

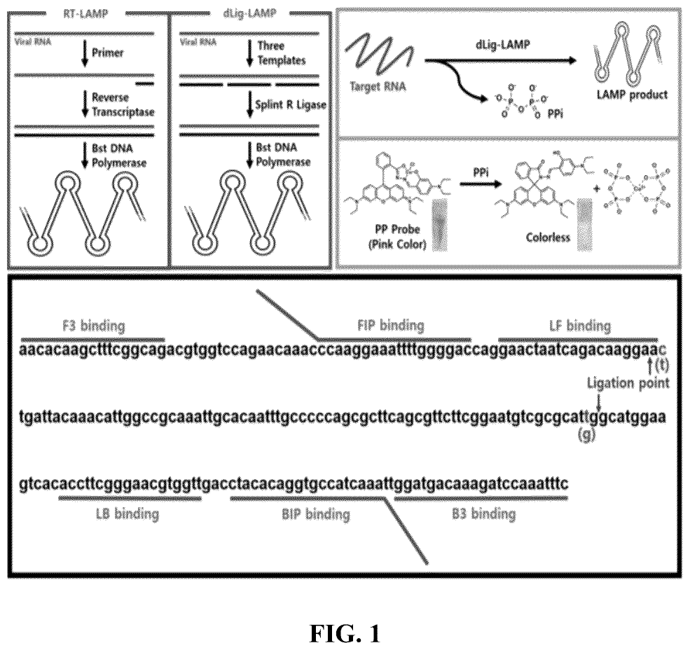

For the LAMP amplification process, six primers (F3, B3, FIP, BIP, LF, LB; Table S1) were designed. To examine the selectivity, not only the target RNA but also one- and two-base-mismatched target RNAs were designed (Table 1). The mismatched points in the target RNA were highlighted with red characters in FIG. 1 (black box). Theoretically, a general RT-PCR or RT-LAMP system is not capable of discriminating a perfectly matched sequence from one- or two-base-mismatched sequences, because the system amplifies the gene with mismatched-sequence amplicons. In contrast, the dLig-LAMP system of the present invention identified mismatched points during the ligation step. In other words, Splint R ligase produced complementary cDNA sequences only in the presence of a perfectly matched target RNA sequence. When one of the ligation templates does not match, the cDNA was not be synthesized. Therefore, it was expected that the selectivity of the dLig-LAMP assay of the present invention would be superior to that of general RT-LAMP or RT-PCR.

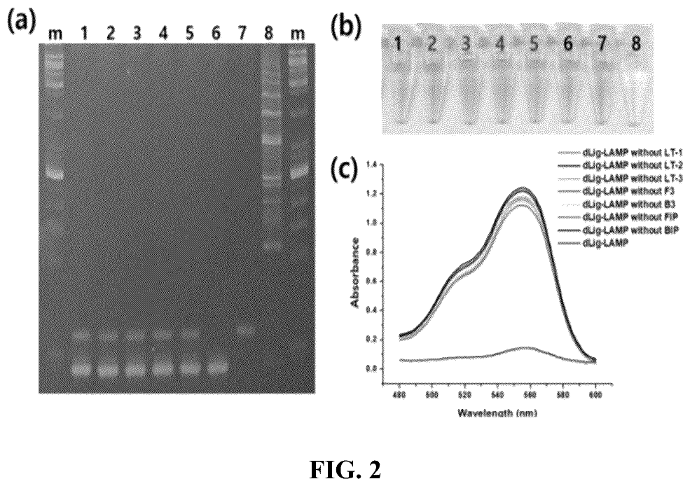

First, it was confirmed whether the dLig-LAMP assay operated in the presence of the SARS-CoV-2 N gene sequence. According to FIG. 2A, it was confirmed that the dLig-LAMP assay functioned for the target SARS-CoV-2 N gene sequence, but not in the absence of any of the primers or templates. The dLig-LAMP system requires six primers and three templates. According to the PAGE images, the negative control lanes (lanes 1 to 7) did not exhibit any amplification patterns, but lane 8, in which all of the primers and templates were present, provided an amplification pattern. Therefore, it was found that dLig-LAMP operated with all of the primers for LAMP and with the three templates, but it did not operated at all when one of them was absent. Next, the possibility of colorimetric detection when using the PP probe to sense the pyrophosphate formed during the DNA amplification was examined (FIG. 2B). The dLig-LAMP assay released an abundance of pyrophosphate during the LAMP DNA amplification. The released pyrophosphate may extract the Cu²⁺ ion from the PP probe, resulting in a color change from pink to colorless, and it was confirmed that such a dramatic color change occurred only in the presence of the SARS-CoV-2 N gene. Similar to the PAGE observations, none of the other samples 1 to 7 resulted in any color change. The absorption spectra exhibited a dramatic decrease in absorption at 555 nm (λₘₐₓ), the characteristic signal of the PP probe, in the presence of the target SARS-CoV-2 N gene (sample 8), but it was confirmed that no such change occurred for any of the other samples (FIG. 2C). Therefore, it was confirmed that when the dLig-LAMP system is combined with the PP probe, the target SARS-CoV-2 N gene may be detected with a colorimetric signal change.

### 2. Experimental Results

### 2-1. Optimization

Three templates (LT1, LT2, LT3) for the synthesis of target cDNA mediated by Splint R ligase (Table 1).

TABLE 1

| Name | Base sequence (5′→3′) | SEQ ID NO. |
|---|---|---|
| F3 | AACACAAGCTTTCGGCAG | SEQ ID NO: 1 |
| B3 | GAAATTTGGATCTTTGTCATCC | SEQ ID NO: 2 |
| FIP | TGCGGCCAATGTTTGTAATCAGCCAAGGAAAT | SEQ ID NO: 3 |
| | TTTGGGGAC | |
| BIP | CGCATTGGCATGGAAGTCACTTTGATGGCACC | SEQ ID NO: 4 |
| | TGTGTAG | |
| LF | TTCCTTGTCTGATTAGTTC | SEQ ID NO: 5 |
| LB | ACCTTCGGGAACGTGGTT | SEQ ID NO: 6 |
| LT-1 | GAAATTTGGATCTTTGTCATCCAATTTGATGGC | SEQ ID NO: 7 |
| | ACCTGTGTAGGTCAACCACGTTCCCGAAGGTG | |
| | TGACTTCCATGC | |
| LT-2 | pho-CAATGCGCGACATTCCGAAGAACGCTGAA | SEQ ID NO: 8 |
| | GCGCTGGGGGCAAATTGTGCAATTTGCGGCCA | |
| | ATGTTTGTAATCAGT | |
| LT-3 | pho-TCCTTGTCTGATTAGTTCCTGGTCCCCAAA | SEQ ID NO: 9 |
| | ATTTCCTTGGGTTTGTTCTGGACCACGTCTGCC | |
| | GAAAGCTTGTGT | |
| Target RNA | aacacaagctttcggcagacgtggtccagaacaaacccaaggaaattttg | SEQ ID NO: 10 |
| | gggaccaggaactaatcagacaaggaactgattacaaacattggccgcaa | |
| | attgcacaatttgcccccagcgcttcagcgttcttcggaatgtcgcgcattgg | |
| | catggaagtcacaccttcgggaacgtggttgacctacacaggtgccatcaa | |
| | attggatgacaaagatccaaatttc | |
| One-based-mi | aacacaagctttcggcagacgtggtccagaacaaacccaaggaaattttg | SEQ ID NO: 11 |
| smatched | gggaccaggaattaatcagacaaggaactgattacaaacattggccgcaa | |
| target RNA | attgcacaatttgcccccagcgcttcagcgttcttcggaatgtcgcgcattgg | |
| | catggaagtcacaccttcgggaacgtggttgacctacacaggtgccatcaa | |
| | attggatgacaaagatccaaatttc | |
| Two-base-mis | aacacaagctttcggcagacgtggtccagaacaaacccaaggaaattttg | SEQ ID NO: 12 |
| matched | gggaccaggaattaatcagacaaggaactgattacaaacattggccgcaa | |
| target RNA | attgcacaatttgcccccagcgcttcagcgttcttcggaatgtcgcgcatgg | |
| | gcatggaagtcacaccttcgggaacgtggttgacctacacaggtgccatca | |
| | aattggatgacaaagatccaaatttc | |

Among them, the LT2 and LT3 templates presented a monophosphate unit at the 5′-end for the ligation event. Templates containing a phosphate modification at the 5′-end also allow the ligation by Splint R ligase to occur in the presence of a perfectly matched target RNA.

For the LAMP amplification process, six primers (F3, B3, FIP, BIP, LF, LB; Table S1) were designed. To examine the selectivity, not only the target RNA but also one- and two-base-mismatched target RNAs were designed (Table 1). The mismatched points in the target RNA were highlighted with red characters in FIG. 1 (black box). Theoretically, a general RT-PCR or RT-LAMP system is not capable of discriminating a perfectly matched sequence from one- or two-base-mismatched sequences, because the system amplifies the gene with mismatched-sequence amplicons. In contrast, the dLig-LAMP system of the present invention identified mismatched points during the ligation step. In other words, Splint R ligase produced complementary cDNA sequences only in the presence of a perfectly matched target RNA sequence. When one of the ligation templates does not match, the cDNA was not be synthesized. Therefore, it was expected that the selectivity of the dLig-LAMP assay of the present invention would be superior to that of general RT-LAMP or RT-PCR.

First, it was confirmed whether the dLig-LAMP assay operated in the presence of the SARS-CoV-2 N gene sequence. According to FIG. 2A, it was confirmed that the dLig-LAMP assay functioned for the target SARS-CoV-2 N gene sequence, but not in the absence of any of the primers or templates. The dLig-LAMP system requires six primers and three templates. According to the PAGE images, the negative control lanes (lanes 1 to 7) did not exhibit any amplification patterns, but lane 8, in which all of the primers and templates were present, provided an amplification pattern. Therefore, it was found that dLig-LAMP operated with all of the primers for LAMP and with the three templates, but it did not operated at all when one of them was absent. Next, the possibility of colorimetric detection when using the PP probe to sense the pyrophosphate formed during the DNA amplification was examined (FIG. 2B). The dLig-LAMP assay released an abundance of pyrophosphate during the LAMP DNA amplification. The released pyrophosphate may extract the Cu²⁺ ion from the PP probe, resulting in a color change from pink to colorless, and it was confirmed that such a dramatic color change occurred only in the presence of the SARS-CoV-2 N gene. Similar to the PAGE observations, none of the other samples 1 to 7 resulted in any color change. The absorption spectra exhibited a dramatic decrease in absorption at 555 nm (λₘₐₓ), the characteristic signal of the PP probe, in the presence of the target SARS-CoV-2 N gene (sample 8), but it was confirmed that no such change occurred for any of the other samples (FIG. 2C). Therefore, it was confirmed that when the dLig-LAMP system is combined with the PP probe, the target SARS-CoV-2 N gene may be detected with a colorimetric signal change.

2-2. Sensitivity and Selectivity with Model Target

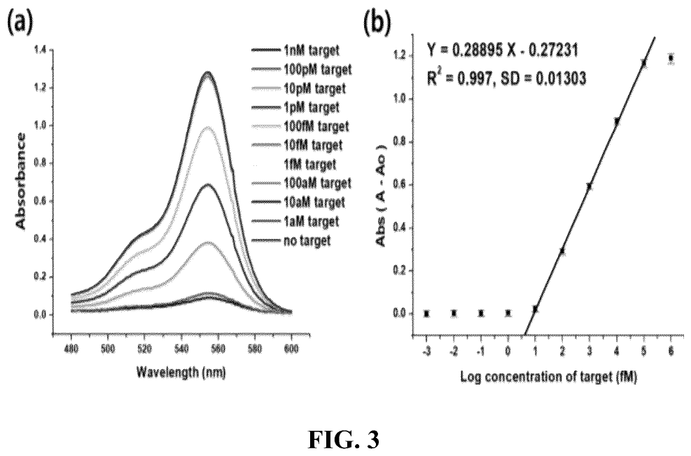

Next, the sensitivity of the dLig-LAMP assay when varying the concentration of the target RNA from 1 aM to 1 nM was measured. FIG. 3A shows the absorbance spectra of the PP probe recorded after performing the dLig-LAMP reaction at various concentrations. The spectra show that the sensitivity of the dLig-LAMP assay depended on the log concentration of the target RNA. To obtain the exact sensitivity, the LOD was measured from concentration-dependent experiments performed in triplicate. From the linear plot of the average absorbance change value obtained at each concentration (FIG. 3B), the LOD value was calculated using the 3σ method [LOD=3×(SD/S), where SD is the standard deviation and S is the slope of the logarithmic plot], and as a result, it was confirmed that the LOD when using the dLig-LAMP assay was 1.36 fM.

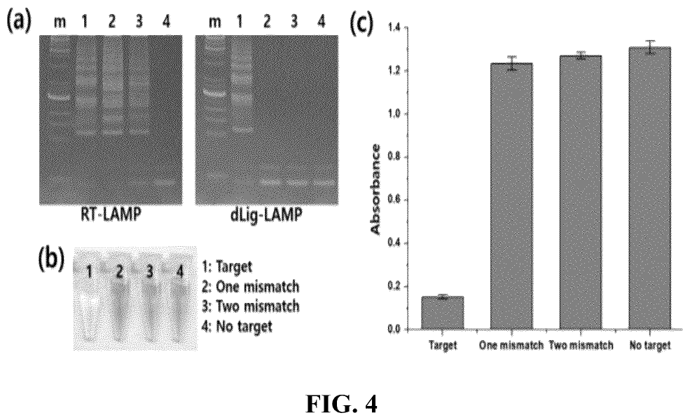

The selectivity of the dLig-LAMP system was much higher than that of the RT-LAMP system, as determined using PAGE (FIG. 4A). The dLig-LAMP assay clearly discriminated the perfectly matched sequence from one to two-base-mismatched sequences, with the appearance of an amplification band. On the contrary, the RT-LAMP assay did discriminate between the perfectly matched and mismatched sequences. All of the sequences, except for the sample obtained without the target, provided the band corresponding to the amplified DNA. It was suspected that the mismatched sequences also bound to the SARS-CoV-2 N gene and underwent reverse transcription to produce cDNA. The dLig-LAMP assay did not produce the cDNA when any mismatches appeared in the template. In the absence cDNA, the LAMP process could not function. When the PP probe was added to the dLig-LAMP assay system, a dramatic color change from pink to colorless was observed only in the presence of the target SARS-CoV-2 N gene (FIG. 4B). FIG. 4C shows the absorbance intensities of the dLig-LAMP/PP probe assays performed with the SARS-CoV-2 N gene, the one-base-mismatched target, and the two-base-mismatched target, and without the target. The dLig-LAMP assay was confirmed to be highly selective in discriminating the mismatched sequences from the perfectly matched target sequence, even when there was only one mismatched base. Although the selectivity of the dLig-LAMP system was higher than that of the general RT-LAMP system, it was considered that it might be difficult to distinguish a mismatch when the mismatch appeared at an irregular position, due to the limited length of the ligation template. In other words, when the template is shortened, the stability of the duplex may be sensitive to differences between the perfectly matched and mismatched sequences, so that it may sufficiently discriminate mismatches from the perfectly matched sequence in all regions.

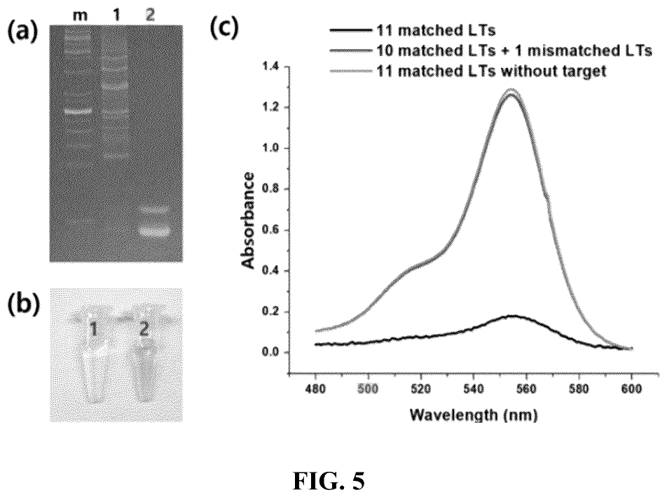

Therefore, the multiple ligation-assisted LAMP reaction was examined using short-ligation templates (FIG. 5). Eleven short-ligation templates (LTs-1 to LTs-11) were designed, and each of the LTs sequences was complementary to the target RNA (Table 2).

TABLE 2

| Name | Base sequence (5′→3′) | SEQ ID NO. |
|---|---|---|
| LTs-1 | AACACAAGCTTTCGGCAGACG | SEQ ID NO: 13 |
| LTs-2 | pho-TGGTCCAGAACAAACCCAAGG | SEQ ID NO: 14 |
| LTs-3 | pho-AAATTTTGGGGACCAGGAACT | SEQ ID NO: 15 |
| LTs-4 | pho-AATCAGACAAGGAACTGATTA | SEQ ID NO: 16 |
| LTs-5 | pho-CAAACATTGGCCGCAAATTGC | SEQ ID NO: 17 |
| LTs-6 | pho-ACAATTTGCCCCCAGCGCTTC | SEQ ID NO: 18 |
| LTs-7 | pho-AGCGTTCTTCGGAATGTCGCG | SEQ ID NO: 19 |
| LTs-8 | pho-CATTGGCATGGAAGTCACACC | SEQ ID NO: 20 |
| LTs-9 | pho-TTCGGGAACGTGGTTGACCTA | SEQ ID NO: 21 |
| LTs-10 | pho-CACAGGTGCCATCAAATTGGA | SEQ ID NO: 22 |
| LTs-11 | pho-TGACAAAGATCCAAATTTC | SEQ ID NO: 23 |
| LTs-6 mismatch | pho-ACATTTTGGCCCCAGCACTTC | SEQ ID NO: 24 |

In addition, one template (LTs-6 mismatch) was modified to contain a mismatched sequence, and when any of the templates was not matched with the target, the selectivity was again examined. Although the multiple ligation-assisted LAMP reaction also operated well, it did not occur when one of the ligation templates was not a perfect match. Therefore, it was confirmed that this method may be useful for increasing the selectivity of the virus detection with sufficiently low rates of false-positives.

2-3. Sensitivity and Selectivity with SARS-CoV-2 and Bacteria

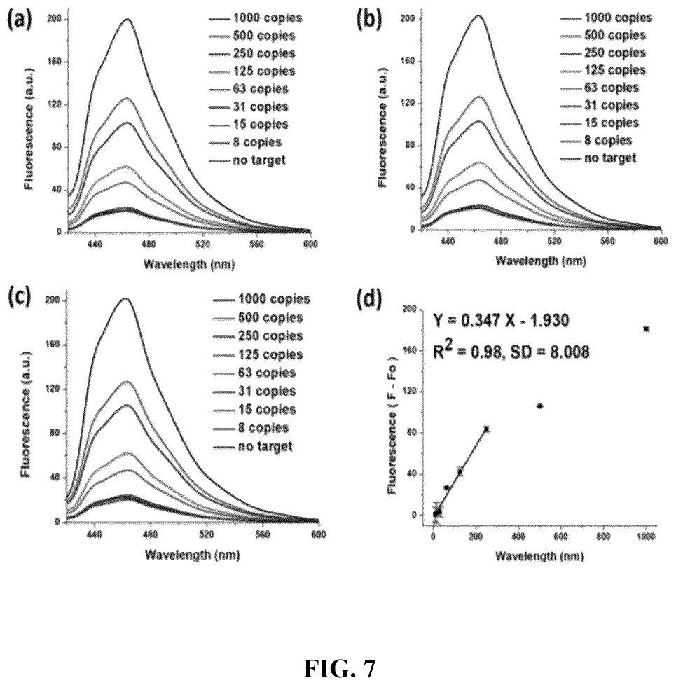

To examine whether the system of the present invention is practically applicable, a sensitivity study was performed using the full genome of SARS-CoV-2 (FIG. 4G). The absorption spectra were measured in experiments performed in triplicate while varying the number of copies of the SARS-CoV-2 full genome from 8 to 1000 copies/rxn. As a result, an LOD (3σ method) of 61.4 copies/rxn was obtained. According to these results, although the detection limit of the system of the present invention is lower than that of the RT-PCR system, it has advantages in that the detection time is shorter (the system of the present invention requires a total of one hour), no heavy instrument is necessary, and treatment may be performed in a simple manner. Thus, it was confirmed that the LOD when using the colorimetric dLig-LAMP system of the present invention is applicable for point-of-care detection of SARS-CoV-2. In addition, the PP probe exhibits fluorescence when the Cu²⁺ ion is lost. Thus, the LOD value of the fluorometric assay was calculated (FIG. 7). As a result, the fluorometric LOD value was found to be 69.2 copies/rxn, indicating that the assay has a similar function to the absorbance assay. Therefore, it was confirmed that the system of the present invention may be used in both absorbance and fluorescence.

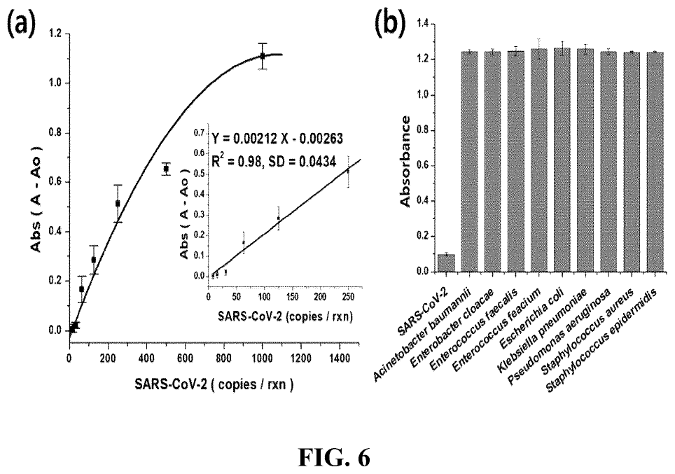

The possibility of cross reactions was examined using nine bacterial genomes (FIG. 6B) that may infect humans and are present in the mucous membranes of the mouth and nose. Actual clinical samples may include these bacterial genomes. When any cross reactions occur due to bacterial genomes that potentially induce false-positive signals, SARS-CoV-2 will not be detected selectively. Therefore, the selectivity of the colorimetric dLig-LAMP system targeting SARS-CoV-2 was examined when these bacteria were present. As a result, none of the nine bacteria reacted with the dLig-LAMP system in the experiments performed in triplicate, and pyrophosphate was not produced. Thus, it was confirmed that the system of the present invention is suitable for use as a point-of-care SARS-CoV-2 detection tool.

### 2-4. Clinical Validation

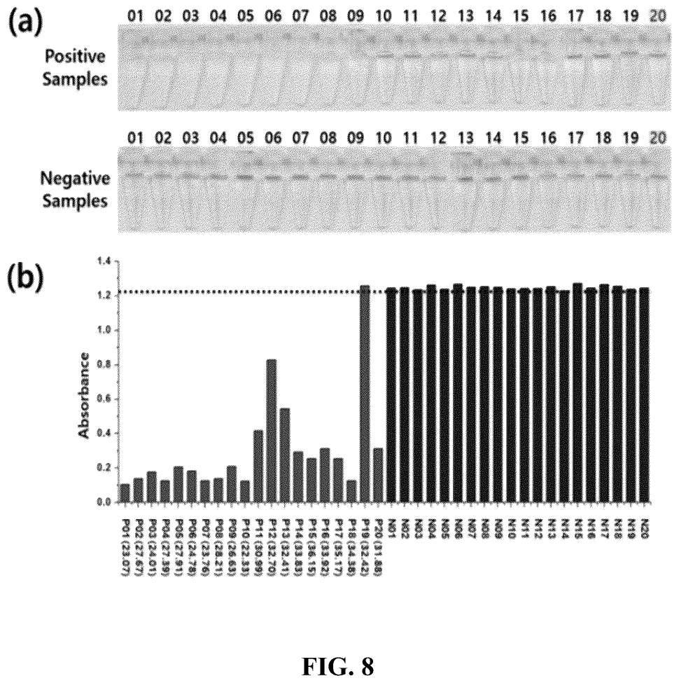

Finally, actual clinical SARS-CoV-2 samples were diagnosed using the dLig-LAMP system with the PP probe (FIG. 8). Twenty positive samples and 20 negative samples were prepared from patients. Many false-negative cases have been reported when RT-LAMP was used to diagnose patients with COVID-19. In contrast, the dLig-LAMP system of the present invention provided 100% true-negatives (negatives classified as negative) through both colorimetric detection and absorbance spectral measurements. In addition, the positive samples provided 95% true-positives (positives classified as positive). At a Ct value below 30, the clinical samples exhibited a strong signal change (complete color change). At a Ct value of 30 or more, the clinical samples exhibited a less signal change compared to the samples with a Ct value less than 30. In addition, the selectivity was confirmed in the case of other types of clinical viral genome. Clinical samples of five different viruses (Influenza A Virus, Influenza B Virus, Respiratory Syncytial Virus A, Respiratory Syncytial Virus B, and Human Rhinovirus) were prepared and tested with SARS-CoV-2 (FIG. S8). As a result, none of the viral genomes other than SARS-CoV-2 reacted with the dLig-LAMP/PP probe system.

According to these results of the clinical validation, it was shown that the selectivity of the dLig-LAMP assays of the present invention was improved compared to that of the general RT-LAMP detection assay. The dual-site ligation route of the present invention to the production of cDNA may be used in some diagnoses that require greater selectivity, and it may also be suitable for point-of-care detection for various RNA-based diagnoses. The procedures are carried out in a simple and rapid (one hour) one-pot reaction.

The present invention has been described with reference to embodiments thereof. Those skilled in the art will understand that the present invention may be implemented in modified forms without departing from the essential characteristics of the present invention. Therefore, the disclosed embodiments should be considered from an illustrative perspective rather than a restrictive perspective. The scope of the present invention is set forth in the claims, not in the foregoing description, and all differences within the scope equivalent thereto should be construed as being included in the present invention.

What is claimed is:

1. A multiple ligation-assisted recombinase polymerase amplification (mLig-RPA) method comprising:
a plurality of template sequences complementary to a part of a base sequence of a target gene;
a ligase;
a loop-mediated isothermal amplification reagent; and
a primer set for loop-mediated isothermal amplification,
wherein the plurality of template sequences are complementary to the entire base sequence of the target gene, when all of the plurality of template sequences are ligated.

2. The composition for detecting a target gene of claim 1, wherein the number of the plurality of template sequences is 2 to 10.

3. The composition for detecting a target gene of claim 1, wherein the target gene consists of 20 to 200 base sequences.

4. The composition for detecting a target gene of claim 1, wherein the ligase is Splint R ligase.

5. The composition for detecting a target gene of claim 1, wherein the primer set for loop-mediated isothermal amplification consists of SEQ ID NOs: 1 to 6.

6. The composition for detecting a target gene of claim 1, wherein the plurality of template sequences complementary to a part of a base sequence of a target gene consist of SEQ ID NOs: 7 to 9.

7. The composition for detecting a target gene of claim 1, wherein the target gene is a SARS-CoV-2 virus-derived base sequence.

8. The composition for detecting a target gene of claim 7, wherein the SARS-CoV-2 virus-derived base sequence is SEQ ID NO: 10.

9. The composition for detecting a target gene of claim 1, further comprising a compound represented by Chemical Formula 1 below:

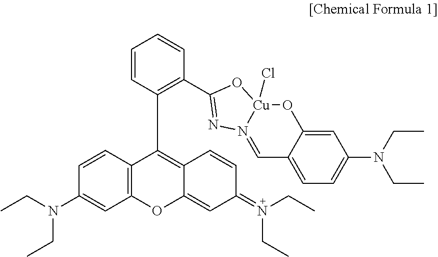

10. An information providing method for determining the presence of a target gene from a subject, comprising:
obtaining a biological sample from a subject;
performing multiple ligation of template sequences by adding a plurality of template sequences complementary to a part of a base sequence of a target gene and a ligase to the sample;
performing an amplification reaction of the multiple ligated template sequences; and
determining that the target gene is present in the subject when the amplification reaction of the template sequences is confirmed.

11. The information providing method for determining the presence of a target gene from a subject according to claim 10, wherein the number of the plurality of template sequences is 2 to 10.

12. The information providing method for determining the presence of a target gene from a subject according to claim 10, wherein the target gene consists of 20 to 200 base sequences.

13. The information providing method for determining the presence of a target gene from a subject according to claim 10, wherein the multiple ligation is performed by Splint R ligase.

14. The information providing method for determining the presence of a target gene from a subject according to claim 10, wherein the amplification reaction is loop-mediated isothermal amplification.

15. The information providing method for determining the presence of a target gene from a subject according to claim 10, wherein the plurality of template sequences consist of SEQ ID NOs: 7 to 9.

16. The information providing method for determining the presence of a target gene from a subject according to claim 10, wherein the target gene is a SARS-CoV-2 virus-derived base sequence.

17. The information providing method for determining the presence of a target gene from a subject according to claim 16, wherein the SARS-CoV-2 virus-derived base sequence is SEQ ID NO: 10.

18. The information providing method for determining the presence of a target gene from a subject according to claim 10, wherein confirmation of the amplification reaction is performed by confirming change in the color of an amplification product before or after the amplification reaction by adding a compound represented by Chemical Formula 1 below: [chemical structure]


SEQUENCE LISTING (24 sequences)

SEQ ID NO: 1 - DNA, 18 bp, synthetic construct
  misc_feature 1..18: note = F3
  source 1..18: mol_type = other DNA
aacacaagctttcggcag

SEQ ID NO: 2 - DNA, 22 bp, synthetic construct
  misc_feature 1..22: note = B3
  source 1..22: mol_type = other DNA
gaaatttggatctttgtcatcc

SEQ ID NO: 3 - DNA, 41 bp, synthetic construct
  misc_feature 1..41: note = FIP
  source 1..41: mol_type = other DNA
tgcggccaatgtttgtaatcagccaaggaaattttggggac

SEQ ID NO: 4 - DNA, 39 bp, synthetic construct
  misc_feature 1..39: note = BIP
  source 1..39: mol_type = other DNA
cgcattggcatggaagtcactttgatggcacctgtgtag

SEQ ID NO: 5 - DNA, 19 bp, synthetic construct
  misc_feature 1..19: note = LF
  source 1..19: mol_type = other DNA
ttccttgtctgattagttc

SEQ ID NO: 6 - DNA, 18 bp, synthetic construct
  misc_feature 1..18: note = LB
  source 1..18: mol_type = other DNA
accttcgggaacgtggtt

SEQ ID NO: 7 - DNA, 77 bp, synthetic construct
  misc_feature 1..77: note = LT-1
  source 1..77: mol_type = other DNA
gaaatttggatctttgtcatccaatttgatggcacctgtgtaggtcaaccacgttcccgaaggtgtgacttccatgc

SEQ ID NO: 8 - DNA, 76 bp, synthetic construct
  misc_feature 1..76: note = LT-2
  source 1..76: mol_type = other DNA
caatgcgcgacattccgaagaacgctgaagcgctgggggcaaattgtgcaatttgcggccaatgtttgtaatcagt

SEQ ID NO: 9 - DNA, 75 bp, synthetic construct
  misc_feature 1..75: note = LT-3
  source 1..75: mol_type = other DNA
tccttgtctgattagttcctggtccccaaaatttccttgggtttgttctggaccacgtctgccgaaagcttgtgt

SEQ ID NO: 10 - DNA, 229 bp, synthetic construct
  misc_feature 1..229: note = Target RNA
  source 1..229: mol_type = other DNA
aacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttc

SEQ ID NO: 11 - DNA, 229 bp, synthetic construct
  misc_feature 1..229: note = One-based-mismatched target RNA
  source 1..229: mol_type = other DNA
aacacaagctttcggcagacgtggtccagaacaaacccaaggaaattttggggaccaggaattaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttc

SEQ ID NO: 12 - DNA, 229 bp, synthetic construct
  misc_feature 1..229: note = Two-base-mismatched target RNA
  source 1..229: mol_type = other DNA
aacacaagctttcggcagacgtggtccagaacaaacccaaggaaattttggggaccaggaattaatcagacaaggaactgattacaaacattggccgcaaattgcacaatttgcccccagcgcttcagcgttcttcggaatgtcgcgcatgggcatggaagtcacaccttcgggaacgtggttgacctacacaggtgccatcaaattggatgacaaagatccaaatttc

SEQ ID NO: 13 - DNA, 21 bp, synthetic construct
  misc_feature 1..21: note = LTs-1
  source 1..21: mol_type = other DNA
aacacaagctttcggcagacg

SEQ ID NO: 14 - DNA, 21 bp, synthetic construct
  misc_feature 1..21: note = LTs-2
  source 1..21: mol_type = other DNA
tggtccagaacaaacccaagg

SEQ ID NO: 15 - DNA, 21 bp, synthetic construct
  misc_feature 1..21: note = LTs-3
  source 1..21: mol_type = other DNA
aaattttggggaccaggaact

SEQ ID NO: 16 - DNA, 21 bp, synthetic construct
  misc_feature 1..21: note = LTs-4
  source 1..21: mol_type = other DNA
aatcagacaaggaactgatta

SEQ ID NO: 17 - DNA, 21 bp, synthetic construct
  misc_feature 1..21: note = LTs-5
  source 1..21: mol_type = other DNA
caaacattggccgcaaattgc

SEQ ID NO: 18 - DNA, 21 bp, synthetic construct
  misc_feature 1..21: note = LTs-6
  source 1..21: mol_type = other DNA
acaatttgcccccagcgcttc

SEQ ID NO: 19 - DNA, 21 bp, synthetic construct
  misc_feature 1..21: note = LTs-7
  source 1..21: mol_type = other DNA
agcgttcttcggaatgtcgcg

SEQ ID NO: 20 - DNA, 21 bp, synthetic construct
  misc_feature 1..21: note = LTs-8
  source 1..21: mol_type = other DNA
cattggcatggaagtcacacc

SEQ ID NO: 21 - DNA, 21 bp, synthetic construct
  misc_feature 1..21: note = LTs-9
  source 1..21: mol_type = other DNA
ttcgggaacgtggttgaccta

SEQ ID NO: 22 - DNA, 21 bp, synthetic construct
  misc_feature 1..21: note = LTs-10
  source 1..21: mol_type = other DNA
cacaggtgccatcaaattgga

SEQ ID NO: 23 - DNA, 19 bp, synthetic construct
  misc_feature 1..19: note = LTs-11
  source 1..19: mol_type = other DNA
tgacaaagatccaaatttc

SEQ ID NO: 24 - DNA, 21 bp, synthetic construct
  misc_feature 1..21: note = LTs-6 mismatch
  source 1..21: mol_type = other DNA
acattttggccccagcacttc